(19) United States
(12) Patent Application Publication
(10) Pub. No.: US 2004/0152774 A1
(43) Pub. Date: Aug. 5, 2004

(54) Method of protecing against tissue extra vasation injury

(76) Inventor: Frederick H. Hausheer, Boerne, TX (US)

Correspondence Address: Thomas J. Dodd, BioNumerik Pharmaceuticals, Inc., Suite 1250, 8122 Datapoint Drive, San Antonio, TX, 78229, US

(21) Appl. No.: 10/357,714

(22) Filed: Feb. 4, 2003

(51) Int. Cl. A61K 31/185; A61K 31/66

(52) U.S. Cl. 514/553; 514/127

(57) ABSTRACT

A method of reducing the risks of tissue extravasation injury associated with intravenous drug therapy is disclosed. The method of this invention includes administering effective amounts of specific sulfur-containing drug agents according to Formula I herein to a patient at risk of extravasation injury.

### FIELD OF THE INVENTION

This invention relates to a method of protecting tissues against injury caused by extravasation of certain drug agents during intravenous administration. The method is especially useful as a prophylaxis treatment for patients undergoing chemotherapy.

### BACKGROUND OF THE INVENTION

Intravenous administration of a number of chemotherapeutic drugs is apt to cause extravasation injury, particularly to tissues in the vicinity of the injection site. Among the agents known to cause extravasation injury are the alkylating agents (primarily the nitrogen mustards); the anthracyclines; most antibiotics; the vinca alkaloids; the epipodophyllotoxins; the platinum agents; amsacrine; mitoxantrone; a number of experimental agents, such as 6-hydroxymethylacylfulvene (MGI 114); and many others.

Extravasation is evidenced by localized pain, burning, stinging, and/or swelling at the injection site. When extravasation injury is observed, the treatment must be halted as soon as possible and accepted countermeasures taken to prevent the spread of tissue destruction. Countermeasures include application of cold packs (in the event of vinca extravasation, Warm packs are applied), administration of topical or subdermal (in some cases intravenous) antidotes, and in severe cases, surgical debridement followed by skin grafting of the affected tissue is necessary.

Extravasation is an occasional problem that occurs during intravenous infusions of drugs. The main cause of extravasation is when the needle dislodges from the vein, usually when the outlet port is blocked or stopped up, thereby causing the fluids being administered to flow into the surrounding tissues. A less than careful insertion of the needle can also result in extravasation, also should the patient shake or experience nervous tremor the needle may be dislodged or come loose from the vein.

The accepted treatment regimens for extravasation injury vary depending upon the identity of the drug that causes the tissue damage. Drug agents currently used as possible antidotes for extravasation injury include dimethyl sulfoxide (DMSO), sodium thiosulfate, phentolamine, glyceryl trinitrate, corticosteroids and hyaluronidase.

### SUMMARY OF THE INVENTION

The method of this invention includes the administration of an effective amount of a formula I compound to a patient at risk of extravasation injury.

R₁—S—R₂—R₃ (I)

wherein

R₁ is hydrogen, lower alkyl or —S—R₂—R₃;

R₂ is lower alkylene, optionally substituted by one or more hydroxy, alkoxy, mercapto, nitro or amino moieties for a corresponding hydrogen atom; and

R₃ is sulfonate or phosphonate; and

pharmaceutically acceptable salts thereof.

### DESCRIPTION OF THE PREFERRED EMBODIMENT

The preferred embodiment herein described is not intended to be exhaustive or to limit the invention to the precise form disclosed. It is chosen and described to explain the principles of the invention and its application and practical use to enable others skilled in the art to best follow its teachings.

The method of this invention has application in the medical field, particularly in the intravenous drug therapy art. As stated above, the method involves the administration of an effective amount of a formula I compound to a patient at risk from extravasation injury, particularly a patient at risk of such injury during intravenous drug therapy.

Administration of the formula I compound is through one of several accepted routes, such as oral, topical or parenteral. In oral administration, the patient at risk from extravasation injury, the formula I compound is contained in a swallowable form, such as a tablet, capsule, caplet, lozenge, soluble powder, or other form suitable for oral administration. For topical administration, the formula I compound is mixed with suitable pharmaceutically acceptable excipients to form a lotion or cream or other topical application form. For intravenous or subcutaneous administration, the formula I compound is dissolved or suspended in a pharmaceutically acceptable solvent for administration.

Timing of the administration of the formula I compound depends on the amount of the formula I compound to be administered, the preferred route of administration, the nature of the primary therapeutic drug, whether the formula I compound is used as a prophylaxis or as an antidote, and other factors common or perhaps unique to each individual situation. When used for prophylactic purposes, the formula I compound is always administered at a time prior to the administration of the primary therapeutic drug agent.

Preferred timing for administration of the formula I compound is from five minutes to one hour prior to commencement of administration of the primary drug. The timing is devised to maximize the concentrations of the formula I compound in surrounding tissues at risk from extravasation, during the time frame when the risk is greatest.

The effective amount of formula I compound to be administered is defined as that amount which effectively reduces tissue injury in the event of an extravasation of the primary drug. Exact amounts will vary from patient-to-patient, and the route of administration and timing will also affect the preferred dosage. Effective oral and parenteral doses may range from as little as 0.1 g/m² up to 80.0 g/m² or higher. Preferred dosage amounts are from 4.0 g/m² to 42.0 g/m², and preferred administration in from five minutes to about one hour prior to commencement of administration of the primary drug.

The formula I compound may also be administered subsequent to the completion of IV drug therapy. Subsequent doses reduce the risk of any delayed symptoms from extravasation that may not manifest during the chemotherapy session. Subsequent doses may be self-administered by the patient as needed if pain or discomfort is noted at or around the injection site. These subsequent doses may take place at regular intervals following the chemotherapy session, every two to every twelve hours, preferably four hours to six hours between doses.

Topical dosage forms are typically more complex, both in formulation and measurement of dose. A number of factors influence dose amounts, including but not limited to the concentration of active ingredient; the drug's absorption rate through the skin into surrounding tissues as well as into the bloodstream; effects of any additives and excipients in the formulation; and others. Due to the ability to apply the drug directly to the target area, drug metabolism and distribution are not as critical as with other dosage forms, and topical dosage amounts are often lower than oral or parenteral dosages of the same drug.

Risk of extravasation injury is highest among patients undergoing intravenous administration of certain oncology drugs. In particular, administration of one or more of the following agents are associated with the risk of extravasation injury: alkylating agents (primarily the nitrogen mustards); the anthracyclines; most antibiotics; the vinca alkaloids; the epipodophyllotoxins; the platinum agents; amsacrine; mitoxantrone; a number of experimental agents, such as 6-hydroxymethylacylfulvene (MGI 114); and many others.

The formula I compound is prepared for administration by commonly known synthetic processes, such as the processes taught in U.S. Pat. No. 5,808,140, incorporated herein by reference. After sterilization, the formula I compound is formulated for administration to the patient, with the preferred formulation dependant on the form of administration.

For intravenous or subcutaneous or subdermal or intradermal administration, the formula I compound is dissolved in a suitable solvent, preferably water. Suitable excipients may also be added to the formulation, as described in U.S. Pat. Nos. 5,789,000; 5,919,816; and 5,866,169, which are incorporated herein by reference.

For oral administration, the formula I compound may be combined with pharmaceutically acceptable fillers and then administered as tablets, caplets, or other swallowable form. Alternatively, the formula I compound may be dissolved or suspended in a pharmaceutically acceptable solvent and encapsulated in a swallowable carrier such as a capsule, a gel cap, or other form, or the formula I compound may be dissolved and then administered as a solution or suspension.

For topical administration, the formula I compound is mixed with pharmaceutically acceptable excipients to produce an elegant formulation designed to deliver the formula I compound to the tissue site surrounding the injection area. The formula I compound is preferably dissolved or suspended in a solvent vehicle, most preferably purified water, prior to addition of the excipients. The pharmaceutically acceptable excipients used to create the formulation may include one or more plasticizers, emulsifiers, emollients, pH adjusters, skin penetration enhancers, surfactants, thickening or thinning agents depending on the desired viscosity and applicability of the formulation, and other ingredients as desired.

Administration of the formula I compound is preferably according to one of the following examples, which are illustrative only and not to be considered as limiting the invention to the described details.

### EXAMPLE 1

### Intravenous Administration

A patient about to undergo IV chemotherapy is administered an intravenous dose of 20 g/m² of a sterile solution of disodium 2,2′-dithiobis ethane sulfonate over 15 minutes by slow drip infusion. 15 minutes after the infusion is completed, the patient begins the chemotherapy. Over the course of the following day, the patient may be given additional infusions of 20 g/m² of disodium 2,2′-dithiobis ethane sulfonate every 4 hours, and the patient's progress is monitored for any signs of extravasation injury.

### EXAMPLE 2

### Oral Administration

A patient about to undergo IV chemotherapy is given an oral dose of 20 g/m² of disodium 2,2′-dithiobis ethane sulfonate in a swallowable carrier. 15 to 45 minutes after taking the dose, the patient begins the chemotherapy. The patient is given, or self-administers additional oral doses every 4 hours, with progress monitoring for site extravasation injury determining how long the patient is given additional doses of drug.

### EXAMPLE 3

### Topical Administration

A patient about to undergo IV chemotherapy has a pharmaceutically elegant topical formulation of disodium 2,2′-dithiobis ethane sulfonate applied to the area and the surrounding areas of the planned injection site. 15 to 45 minutes following completion of the application of the formulation, the patient begins the chemotherapy. Further applications of the topical formulation, both during and after the chemotherapy session, may be applied as desired or deemed necessary by the attending health care professional.

### EXAMPLE 4

### Direct Injection Administration

A patient about to undergo IV chemotherapy is administered a dose of a sterile solution or suspension of 1%-75% w/w disodium 2,2′-dithiobis ethane sulfonate by direct tissue injection (subcutaneous, subdermal and/or intradermal) approximately 15 to 45 minutes prior to beginning of the chemotherapy treatment.

It should be noted that while the term chemotherapy is used extensively throughout this specification, that term is to be accorded its broad-based definition as “the use of chemical agents in the treatment or control of disease or mental illness” rather than a narrower colloquial use of the term that associates chemotherapy with cancer treatments.

It should be further noted that combinations of the three recited methods of administration may be practiced according to the teachings of this invention. For example, a patient may receive a pretreatment dose (by any accepted route of administration) of formula I compound prior to beginning chemotherapy, and then be given oral dosages or topical formulation to take home, along with instructions to self-administer or apply the doses following the chemotherapy session should minor discomfort occur around the injection site. Allowing the patient to self-administer subsequent doses aids in convenience and independence for the patient and builds self-esteem, often an important psychological factor for patients who require chemotherapy for any serious or life-threatening illness.

The above description has been presented for illustrative purposes to enable those skilled in the art to understand its teachings, and is not to be considered as limiting the scope of the invention to the precise details herein recited, which scope is defined in the foregoing claims.

I claim:

1. A method for reducing or preventing extravasation injury to bodily tissue in a patient at risk of extravasation injury, said method comprising administering to said patient a therapeutic amount of a formula I compound:
R₁—S—R₂—R₃ (I)
wherein
R₁ is hydrogen, lower alkyl or —S—R₂—R₃;
R₂ is lower alkylene, optionally substituted by one or more hydroxy, alkoxy, mercapto, nitro or amino moieties for a corresponding hydrogen atom; and
R₃ is sulfonate or phosphonate; and
pharmaceutically acceptable salts thereof.

2. The method of claim 1 wherein the therapeutic amount of the formula I compound is from 0.1 g/m² to 80.0 g/m².

3. The method of claim 1 wherein the risk of extravasation injury to the patient is from intravenous drug therapy.

4. The method of claim 3 wherein the therapeutic amount of the formula I compound is administered from 5 minutes to one hour prior to beginning the intravenous drug therapy.

5. The method of claim 1 wherein R₂ is ethylene, R₃ is sulfonate and R₁ is —S—R₂R₃.

6. The method of claim 4 wherein the therapeutic amount is from 4.0 g/m² to 42.0 g-m².

7. The method of claim 4 wherein the therapeutic amount is administered from 25 minutes to 30 minutes prior to beginning the intravenous drug therapy.

8. The method of claim 4 wherein the therapeutic amount is administered at intervals subsequent to the completion of the intravenous drug therapy.

9. The method of claim 8 wherein the intervals are every two to twelve hours following completion of the intravenous drug therapy.

10. The method of claim 4 wherein the formula I compound is administered intravenously.

11. The method of claim 4 wherein the formula I compound is administered subcutaneously.

12. The method of claim 4 wherein the formula I compound is administered topically.
